(19) United States
(12) Patent Application Publication
(10) Pub. No.: US 2021/0389327 A1
(43) Pub. Date: Dec. 16, 2021

(54) Extracelluar Vesicle Biomarkers for Bladder Cancer

(71) Applicant: Tymora Analytical Operations, Inc., West Lafayette, IN (US)

(72) Inventors: Weiguo A. Tao, West Lafayette, IN (US); Anton Ilyuk, West Lafayette, IN (US)

(21) Appl. No.: 17/346,761

(22) Filed: Jun. 14, 2021

Related U.S. Application Data
(60) Provisional application No. 63/038,151, filed on Jun. 12, 2020.

(51) Int. Cl. G01N 33/574

(52) U.S. Cl. CPC G01N 33/57488; G01N 33/57407

(57) ABSTRACT

Methods and products for the identification and detection of new bladder cancer biomarkers based on proteins and protein phosphorylation in urinary extracellular vesicles.

### CROSS-REFERENCE TO RELATED APPLICATION

This U.S. patent application claims priority to U.S. Provisional Application No. 63/038,151 filed Jun. 12, 2020, to the above-named inventors, the disclosure of which is considered part of the disclosure of this application and is hereby incorporated by reference in its entirety.

### FEDERALLY SPONSORED RESEARCH OR DEVELOPMENT

The research was supported by the NIH grant R44CA239845.

### SEQUENCE LISTING, A TABLE, OR A COMPUTER PROGRAM

Not applicable.

### FIELD OF THE INVENTION

This invention relates generally to a method to isolate proteins and phosphoproteins from biofluids, such as urine, for biomarker discovery or for clinical detection. More particularly, this invention relates to non-invasive early disease diagnosis, disease monitoring and disease classification. In one aspect, this invention relates to unique proteins and phosphoproteins capable of differentiating bladder cancer urine from healthy urine and inflammation control urine. In another aspect, this invention relates to early-stage detection of bladder cancer by urine test.

### BACKGROUND

Bladder cancer is the most common cancer of the urinary tract, affecting close to 400,000 people worldwide (72). Despite not being the highest-incidence, it is the most expensive cancer to treat per patient in the U.S. due to the necessary on-going treatment and monitoring with a re-occurrence rate of 50-80% (73). Most of the current bladder cancer marker tests depend on invasive cystoscopy or the presence of exfoliated cancer cells in urine. The typical sensitivity for current tests is 40-80% (74,75), as a result, low tumor burden (low-grade tumors), tumor heterogeneity, or tumor cells that do not exfoliate into urine leave room for improvement in early detection. While some urine-based tests have been approved by the FDA, none of them are routinely used or incorporated into clinical guidelines due to opportunities for improvement in sensitivity (76). Recent studies found that when given a choice between a bladder cancer molecular marker test with <90% sensitivity or cystoscopy (the invasive current standard test for bladder cancer detection), the patients and urologists chose the invasive cystoscopy (77). So, in order to fulfill the market need, a new non-invasive cancer test needs to perform with >90% sensitivity.

Currently the most widespread method for clinical cancer profiling and diagnosis involves a tumor biopsy, an invasive and painful procedure, and one that certainly is impractical for early-stage detection. As cancer becomes a more chronic disease that requires active monitoring over longer periods of time, tissue biopsies on a continuous basis are no longer a realistic scenario. As a result, “liquid biopsies”—analysis of biofluids such as plasma, serum, urine—have gained much attention as a potentially useful source of diagnostic biomarkers. Liquid biopsies offer numerous advantages for a clinical analysis, including non-invasive collection, a suitable sample source for longitudinal disease monitoring, better screenshot of tumor heterogeneity, higher stability and sample volumes, faster processing times, lower rejection rates and cost. However, the most common focus of liquid biopsy—CTCs and ctDNA—still have room for improvement. Improvements can be made with the heterogeneity and extreme rarity of the circulating tumor cells (CTCs) (1). Similarly, circulating DNA (ctDNA) is highly fragmented and exists in very small, often not detectable amounts. This makes opportunity for early disease detection tests development. For example, in bladder cancer, CTCs were found in only 23% of the preoperative patients and did not have any predictive value for survival (2). As a result, only a limited number of effective clinical CTC or ctDNA diagnostics tests are currently on the market. To date, only two such in vitro diagnostics (IVD) tests have been approved by the FDA—Janssen Diagnostics' CellSearch and Cobas EGFR Mutation Test. However, negative results from these tests still require a follow-up biopsy. The lack of other approvals over the past decade further highlights the need for non-invasive early cancer detection tests with high sensitivity.

A new field has generated a lot of interest over the past few years—profiling of cell-secreted extracellular vesicles (EVs). EVs offer all the same attractive advantages of a liquid biopsy offered by CTCs and ctDNA. These generally include smaller size exosomes derived from multivesicular endosome-based secretions, and microvesicles (MVs) derived from the plasma membrane (3-5). The EVs provide an effective and ubiquitous method for intercellular communication and removal of excess materials and are utilized by every cell type studied to date. As these are shed into every biological fluid and embody a good representation of their parent cell, analysis of the EV cargo has great potential for biomarker discovery and disease diagnosis (6). Notably, researchers have also found many differentiating characteristics of the cancer cell-derived cargo, including gene mutations, active miRNA and proteins, which possess metastatic properties (7-11).

Particularly promising are the findings that these EV-based disease markers can be identified well before the onset of symptoms or physiological detection of a tumor. This makes them favorable candidates for early-stage cancer and other disease detection. In addition, EVs are membrane-covered nanoparticles, which protects the inside contents from external proteases and other enzymes (12-14). Applicants reason that these features make EVs a promising source to advance proteins and phosphoproteins as disease markers, considering that many phosphorylation events directly reflect molecular and physiological status of a tumor (15, 16). Despite some encouraging success in the analysis of exosomes for DNA, RNA and protein content, the methods and data for examination of EV phosphoproteomes are not as far along in development. The studies carried out to date include either data from urine, only 14 phosphoproteins identified from 400 mL urine (17), or 82 phosphoproteins identified through analysis of cell culture media, which may not be directly relevant to clinical biomarkers (18). Besides Applicants' recent publications (19, 20, 80), no other fruitful phosphoprotein analysis studies using plasma, urine or cerebrospinal fluid (CSF) are known.

Current EV/Exosome Analyses.

Given the immaturity of EV analysis, a standardized method for collecting and processing EVs has not yet been developed (21). Most studies rely on differential centrifugation, with ultracentrifugation (UC) as the final step. However, this approach has room for advancement for use in a clinical setting due to opportunities for improvement in reproducibility (11, 22). In addition, multiple publications have shown that the exosome recovery rate after ultracentrifugation is only about 5-25% (23-25). Several other groups have published and commercialized new methods for EV isolation, which include polymer-induced precipitation (26, 27), antibody-based capture (28, 29), affinity filtration (30), size-exclusion chromatography (25, 31), among others. Each one has its own opportunities for enhancement, including recovery rate (usually similar or less than ultracentrifugation) and contamination levels (22, 24, 25, 30, 32-37). While these can certainly be used as a faster alternative to UC, at 5-25% published yields, their efficiency of isolation still leaves much room for improvement. High levels of free protein, which may not be a major concern for RNA/DNA analysis (focus of the majority of exosome researchers), provides another avenue for development of the current methods for the proposed phosphoproteome analysis. While genetic testing is very valuable, it could greatly benefit from an additional layer of biological information.

The ability to detect the genome output—active proteins—can provide useful real-time information about the organism's physiological functions and disease progression, particularly in cancer. Oncologists understand the value of protein testing and immunoassays and can easily interpret the results. Compared to gene panel testing, immunoassays are also relatively inexpensive and are more likely to get reimbursed. Nonetheless, the genetic screens are currently dominating the new biomarkers market and the area of molecular diagnostics because the technology for genomic readout and reproducible quantitation is readily available and highly robust.

Protein phosphorylation is a key control mechanism for cellular regulatory pathways, and one often targeted by drug developers to create inhibitors that block signaling pathways involved in cancer and other diseases. However, due to active phosphatases in biofluids, there are few detectable phosphoproteins available for disease status analysis. No successful urine phosphoproteomics results have been reported, besides a recently accepted manuscript (20).

### BRIEF SUMMARY OF THE INVENTION

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

Motivated by the urgent need to develop better biomarkers for early non-invasive diagnosis of bladder cancer, we implemented the approach to discover EV biomarkers directly from urine samples. We applied Extracellular Vesicles total recovery and purification (EVtrap) EV enrichment method to urine samples to isolate EVs for subsequent liquid chromatography—mass spectrometry analysis. EVtrap enables the capture of EVs onto functionalized magnetic beads modified with a combination of hydrophilic and lipophilic groups that have a unique affinity toward lipid-coated EVs (Wu et al. 2018). Over 95% recovery yield can be achieved by EVtrap with less contamination from soluble proteins, a significant improvement over current commercially available methods as well as ultracentrifugation.

Processing and enrichment of EVs through EVtrap enabled the removal of soluble proteins, retaining vesicle associated proteins which are more stable in circulation and have enhanced signals from cancer tissues. The protein profiles in EV concentrates are different from protein profiles naturally occurring in patient urine.

In one aspect, this disclosure is related to a robust method for the identification and detection of new biomarkers based on proteins and protein phosphorylation—a true measure of dynamic activity and cellular signaling, for the purposes of disease diagnosis, prognosis, detection, monitoring, patient stratification, drug response analysis, therapy selection, or the like.

The proposed method introduces a novel platform technology to isolate proteins and phosphoproteins from biofluids, such as urine, for biomarker discovery or for clinical detection.

In another aspect, this disclosure is related to a method that successfully demonstrates the feasibility of developing biofluid-derived EV phosphoproteins for cancer profiling. It has tremendous transformative potential for early cancer diagnosis, monitoring and classification based on actual activated pathways using urine as the source.

Further, once fully established, the method of the present disclosure can be implemented by scientists worldwide to analyze the direct signaling networks for a cancer of interest in a non-invasive manner.

Furthermore, once fully established, these new biomarkers can be employed either isolated or as part of a panel of biomarkers as a liquid biopsy in clinical scenarios: (1) as a surveillance test in high-risk patients, such as those with high-risk cystic diseases, hereditary risk of cancer, among others or (2) as a liquid biopsy for the longitudinal monitoring of treatment response in patients with already established cancer diagnosis.

In yet another aspect, this disclosure relates to a biomarker panel for detection and monitoring of bladder cancer. The approach will enable a truly non-invasive test and the first example of using phosphoproteins for early cancer diagnostics, especially in liquid biopsy setting.

Still further, it is envisioned to further apply this innovative procedure to validate and fully develop pre-determined biomarkers panels.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1A is the comparison between ultracentrifugation (UC) and EVtrap for exosome capture, illustrated by the detection of CD9 exosome marker using Western Blot (WB).

FIG. 1B is the comparison between ultracentrifugation (UC) and EVtrap for exosome capture, illustrated by the quantitation of the WB data in FIG. 1A as a percent recovery from the control sample (n=5).

FIG. 2A is the quantitative exosome capture comparison by CD9 Western Blot between ultracentrifugation (100K UC), EVtrap and three commercial methods.

FIG. 2B is the silver stain total protein contamination comparison of the same samples from FIG. 2A.

FIG. 3 is the test of EVtrap procedure reproducibility carried out by two researchers over 5 days.

FIG. 4A is the LC-MS total proteome analysis of 100K UC and EVtrap samples, illustrated by (A) the quantitation of 13 common exosome proteins and (B) for 5 free urine proteins.

FIG. 4B is the LC-MS total proteome analysis of 100K UC and EVtrap samples, illustrated by (A) the quantitation of 13 common exosome proteins and (B) for 5 free urine proteins.

FIG. 4C is the LC-MS total proteome analysis of 100K UC and EVtrap samples, illustrated by the fold increase in total proteome intensity of known exosome markers compared to UC sample.

FIG. 5A is the LC-MS phosphoproteomic analysis of 100K UC and EVtrap samples, illustrated by the total number of unique phosphopeptides and phosphoproteins identified.

FIG. 5B is the LC-MS phosphoproteomic analysis of 100K UC and EVtrap samples, illustrated by the fold increase in total phosphoproteome intensity from FIG. 5A LC-MS data (EVtrap vs. UC).

FIG. 6A is the total quantitative data of identified and quantified proteins and phosphoproteins, with the inclusion of proteins that are increased at least 4-fold in bladder cancer urine compared to healthy and inflammation controls.

FIG. 6B is the quantitative data of total EV markers, proteins and phosphoproteins that are increasing in bladder cancer urine compared to healthy and inflammation controls.

FIG. 6C is the log-scale heatmap analysis of select proteins capable of differentiating bladder cancer urine from healthy and inflammation controls.

FIG. 7 is the box-and-whisker plots for select proteins and phosphoproteins capable of differentiating bladder cancer urine from healthy and inflammation controls (log 2 intensity scale). Horizontal line represents Not Detectable (N.D.) in those samples.

FIG. 8 is the ROC curve for Protein Marker A of FIG. 7.

FIG. 9 is the illustration of the EVtrap magnetic capture of EVs.

FIG. 10 is the box-and-whisker plots for reverse phase protein assay (RPPA) data of selected 4 protein markers capable of differentiating bladder cancer urine from healthy and inflammation controls (normal urine n=24; bladder cancer urine n=20). The data was normalized to the CD9 RPPA signal within each sample.

FIG. 11A is the volcano plot analysis of urine EV proteins upregulated in bladder cancer urine compared to healthy controls.

FIG. 11B is the log-scale heatmap analysis of select proteins capable of differentiating bladder cancer urine from healthy controls.

FIG. 11C is the volcano plot analysis of urine EV phosphoproteins upregulated in bladder cancer urine compared to healthy controls.

FIG. 11D is the log-scale heatmap analysis of select phosphoproteins capable of differentiating bladder cancer urine from healthy controls.

FIG. 12 is the box-and-whisker plots for select proteins and phosphoproteins capable of differentiating bladder cancer urine from healthy controls (log 2 intensity scale).

FIG. 13A shows Ingenuity Pathway Analysis (IPA) of the bladder cancer urine EV phosphoproteomics data, revealing the top cancer networks upregulated.

FIG. 13B shows IPA ontology illustration of the upregulated and downregulated proteins and phosphoproteins from our data known to be linked to bladder cancer.

FIG. 14A is the volcano plot analysis of urine EV proteins upregulated and downregulated in bladder cancer urine compared to healthy controls from the new validation cohort of 176 patients.

FIG. 14B is the log-scale heatmap analysis of select proteins capable of differentiating bladder cancer urine from healthy controls from the new validation cohort of 176 patients.

FIG. 15 shows the accuracy of the training and test sets of samples for top bladder cancer biomarkers validated from the new 176-patient cohort of samples.

FIG. 16 shows the ROC curve analysis of the top markers validated in this experiment from the new 176 cohort group of patients.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

Unless otherwise stated, a reference to a compound or component includes the compound or component by itself, as well as in combination with other compounds or components, such as mixtures of compounds.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

All publications, patents and patent applications cited herein, whether supra or infra, are hereby incorporated by reference in their entirety to the same extent as if each individual publication, patent or patent application was specifically and individually indicated to be incorporated by reference.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

Extracellular Vesicles Total Recovery and Purification (EVtrap) Technology

Total EV and/or exosome capture and purification has been the focus of many recent studies, with particular consideration toward simple and easy protocol. The vast majority of the exosome analysis projects are based on the differential centrifugation. Although viable, this approach leaves room for improvements for the detection of EV biomarkers in a high-throughput automatable environment. Here, this disclosure introduces a novel non-antibody affinity beads-based capture method for effective EV isolation, termed EVtrap (Extracellular Vesicles total recovery and purification), directly from urine. It enables purification of the complete EV profile based on the lipid bilayer structure of these vesicles and the unique combination of the hydrophilic and aromatic lipophilic groups on the synthesized beads. The introductory manuscript of this technology was published in September 2018 (20).

Comparison of EV capture efficiency by Western Blot.

For the initial method validation, this disclosure compared the EVtrap approach with the “gold standard” ultracentrifugation method using 500 μL of urine for each test.

After the ultracentrifugation step (100K UC), the pellet was boiled and loaded on the gel directly or washed with PBS once or twice and centrifuged again. The supernatant from 100K UC step was concentrated and loaded on the gel in the same proportion.

The EVtrap method was carried out also using 500 μL urine: after 1-hour incubation, the supernatant (unbound fraction) was collected, concentrated and loaded on the gel. The captured EVs were eluted by incubation for 10 minutes with triethylamine. For recovery yield quantitation: the complete EV (exosome) population from 500 μL urine was concentrated and used as the exosome control.

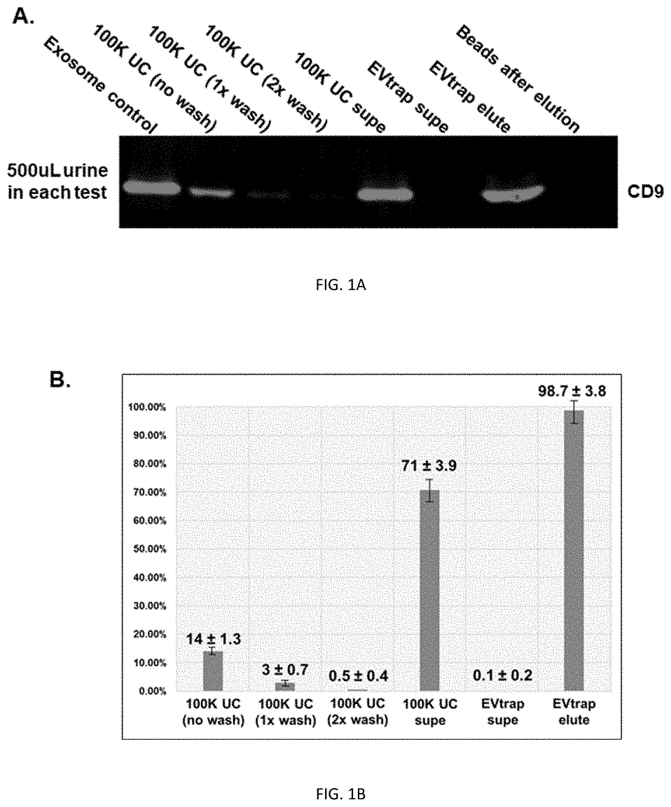

All samples described above were loaded on the same gel and detected by Western Blot using a primary antibody for CD9 (common exosome marker). This experiment was carried out 5 separate times. A representative Western Blot is shown in FIG. 1A, and the quantitative values for each CD9 band signal are listed in the bar graph in FIG. 1B. As the results show, ultracentrifugation step indeed captures only a portion of the exosomes—14% on average—a recovery rate similar to other studies (23, 24). Detection of the UC supernatant further confirmed the incomplete capture, as it is expected to see a large percentage of EVs remaining in the supernatant (38). In contrast, the EVtrap method resulted in no detectable CD9-containing exosomes in the supernatant (unbound fraction), with ˜99% of the exosomes being captured and recovered. As quantified in FIG. 1B, the EVtrap capture and elution reproducibility is outstanding, producing a standard deviation of 3.8%.

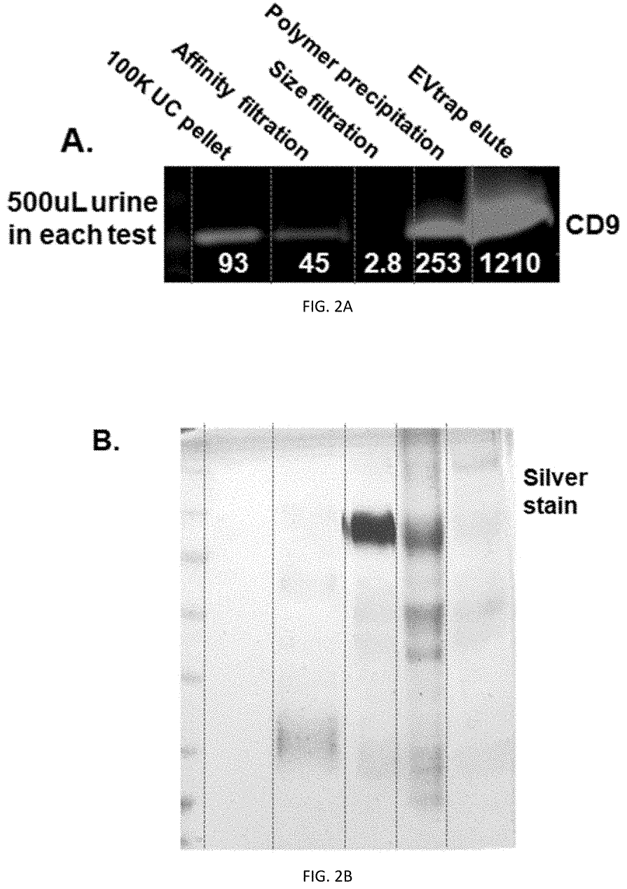

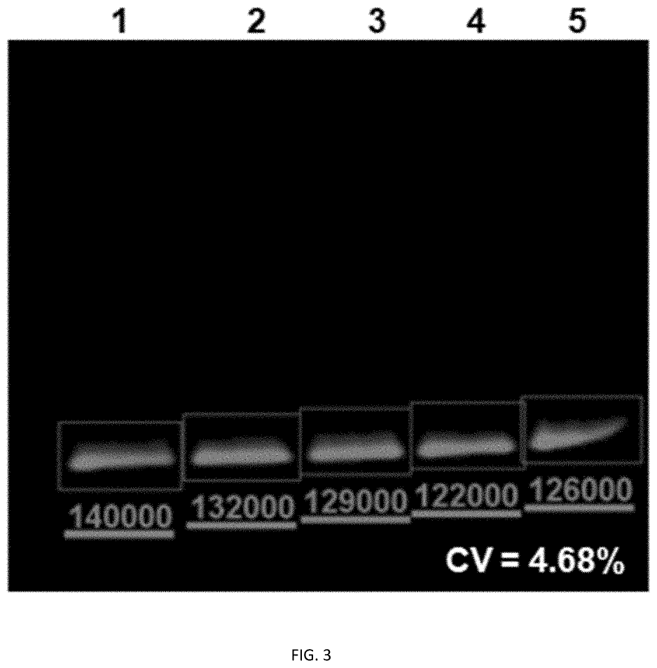

For additional recovery and purity assessment, besides ultracentrifugation, this disclosure also sought to compare other frequently used approaches. This disclosure used three common commercially available methods: including Qiagen's membrane affinity spin method, Hitachi's size-based filtration tube and Invitrogen's polymer-based exosome precipitation. Direct urine was used in each case and 500 μL equivalent was run after capture on two different gels and detected by anti-CD9 antibody (FIG. 2A) or silver stain for purity assessment (FIG. 2B). As the results demonstrate, the alternative methods produced somewhat similar exosome recovery signal compared to 100K ultracentrifugation, matching the previously published results for these methods. When compared to 100K UC pellet, the polymer-based EV precipitation even produced 2.5× higher exosome yield, although the contamination level was also much higher (FIG. 2B). Nonetheless, EVtrap produced significantly higher exosome recovery yield, with lower level of contamination, compared to the other approaches.

In order to enable reliable analysis of clinical samples, the method must be highly reproducible by different users and over time. This disclosure tested the coefficient of variation (CV) of EVtrap capture by running 5 separate experiments using 0.5 mL urine on 5 different days carried out by two independent researchers. The eluted samples were then loaded on the same gel and the exosomal CD9 signal was quantified. FIG. 3 shows <5% CV for EVtrap isolation, demonstrating outstanding day-to-day reproducibility, as is necessary for clinical sample analysis.

While Western Blot-based detection (as used in preliminary evaluation studies) does allow simple analysis of EV markers, there is opportunity for improvement since it tends to work for a few targets at a time, with good antibodies available. Mass spectrometry (MS) analysis enables the detection and quantitation of hundreds or thousands of proteins in a single experiment, while uncovering previously unknown targets. Hence, MS is the method of choice for current cancer biomarker discovery efforts, often coupled with liquid chromatography (LC).

Comparison of EV capture efficiency by LC-MS.

For the LC-MS analysis, this disclosure used 200 μL of urine as the starting material. As a control, ultracentrifugation (100K UC) pellet was used directly for protein extraction. The EVtrap method was then carried out on the supernatant from the 100K UC sample to analyze the exosomes left after the ultracentrifugation step. For the sample comparison, the EVtrap method was also carried out on the 10K supernatant and on 200 μL direct urine.

Using a single 90-min LC-MS gradient, the EVtrap method as disclosed herein was able to identify over 16,000 unique peptides from approximately 2,000 unique proteins. By comparison, ultracentrifugation method produced approximately 7,200 unique peptides from approximately 1,100 unique proteins.

EVtrap Capture Efficiency.

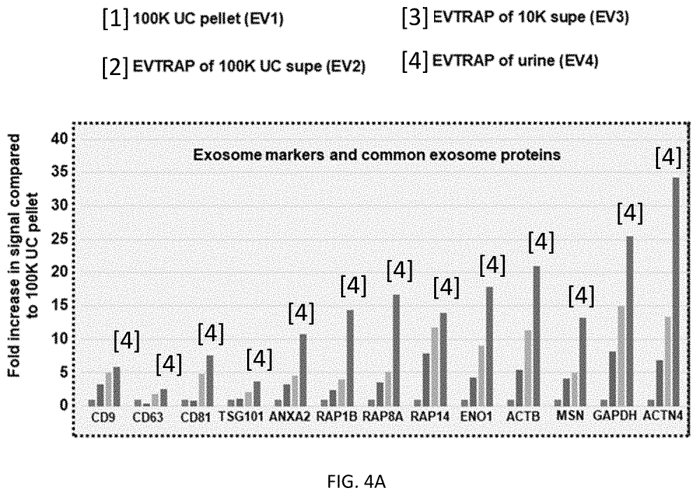

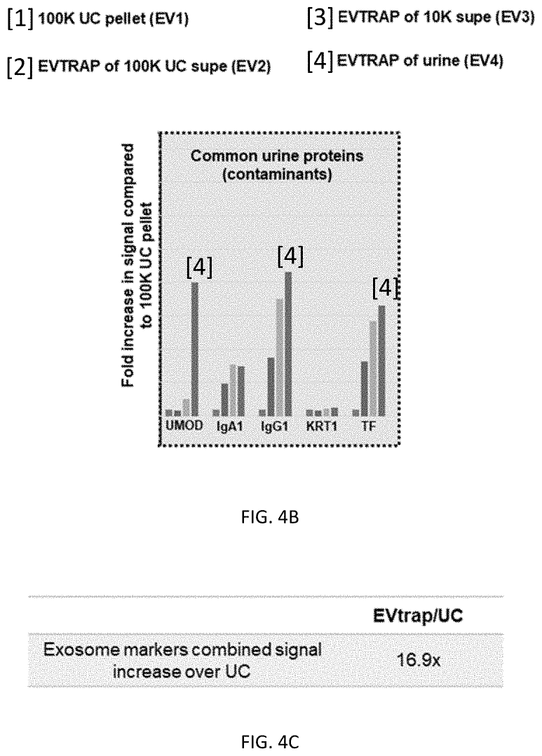

This disclosure further utilized label-free quantitation to compare all of the proteins identified by each method. In this experiment, this disclosure identified and quantified 94 out of 100 common exosome markers published in ExoCarta (39-41). All of them showed a significant increase after EVtrap capture compared to ultracentrifugation. This is noteworthy because many other studies have shown that different methods enrich different exosome populations with various success rates (34, 42). With EVtrap, it appears that the complete EV profile is recovered. As an example of the data, this disclosure listed a few common exosome markers in a bar chart in FIGS. 4A-B. The average increase of all detected exosome markers captured by EVtrap is almost 17-fold higher compared to the UC sample (FIG. 4C). For comparison, this disclosure also quantified any contamination free urine proteins. As shown, highly abundant urine proteins were detected at the levels similar to exosome markers, indicating generally good specificity of EVtrap to enrich EVs with low contamination. It is envisioned that the contamination levels can be further reduced by the extensive washing steps.

EVtrap and Phosphoproteome Analysis.

With the ability of EVtrap to enable improved LC-MS analysis of urinary EVs, this disclosure further sought to apply this approach for phosphoproteome analysis. Despite the increasing interest in biofluid EVs, methods for EV phosphoproteome analysis provide opportunity for improvement in reporting, except a couple of recent publications (19, 20). Indeed, this disclosure's preliminary data have demonstrated that the suggested published EV workflows (43-47) provide opportunities for improvement for subsequent phosphoproteome analysis. It is therefore understood that other methods for EV isolation may be utilized when optimized, such as the commonly used commercial EV isolation methods: Qiagen ExoEASY (membrane filtration), Cell Guidance System Exo-spin (size-exclusion chromatography (SEC)), Thermo Fisher Total Exosome Isolation Reagent (TEIR) (precipitation), JSR Life Sciences ExoCAP (antibody immunoprecipitation (IP)) and 101Bio PureExo kit (precipitation).

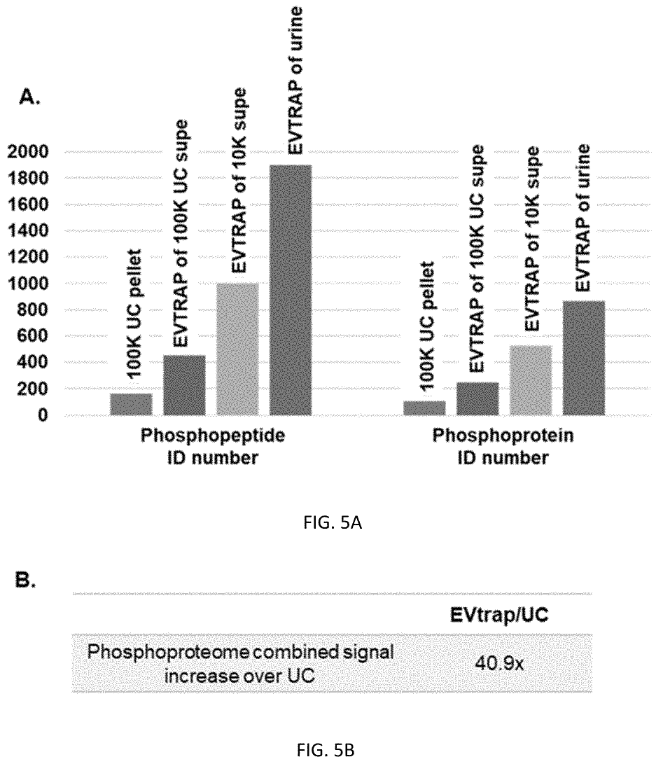

For preliminary phosphoproteomic analyses, this disclosure used 10 mL of urine for each treatment, including EVtrap and ultracentrifugation (100K UC). FIG. 5A shows the phosphoproteome data identified. The UC sample produced 165 unique phosphopeptides from 105 unique phosphoproteins, which seems to be a higher urine phosphoproteome ID number than reported by others. However, when EVtrap was used for capture, this disclosure saw a statistically significant increase in phosphoproteome identification levels. This disclosure identified almost 2,000 unique phosphopeptides from over 860 unique phosphoproteins using only 10 mL of urine and a single 60-min LC-MS run. Most phosphoproteins were not detected by MS after ultracentrifugation. FIG. 5B lists the total increase in signal of the identified phosphoproteins, demonstrating approximately 41-fold increase in EV phosphoproteome signal after EVtrap capture compared to UC. These data show that EVtrap can process urine directly, a highly useful feature for routine clinical analysis. With 10-mL urine being sufficient to identify hundreds of phosphoproteins, the volume is also convenient.

### Example 1: Biomarker Discovery for Bladder Cancer

EVtrap capture was utilized for urine EV proteome and phosphoproteome analysis. This disclosure utilized the EVtrap method for the discovery of novel urinary biomarkers from bladder cancer patients, for the purposes of bladder cancer diagnosis, prognosis, detection, monitoring, patient stratification, drug response analysis, therapy selection, or the like. Here, both urine EV proteomics and phosphoproteomics were carried out to examine both sets of potential biomarkers.

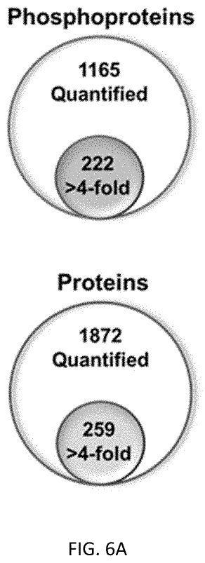

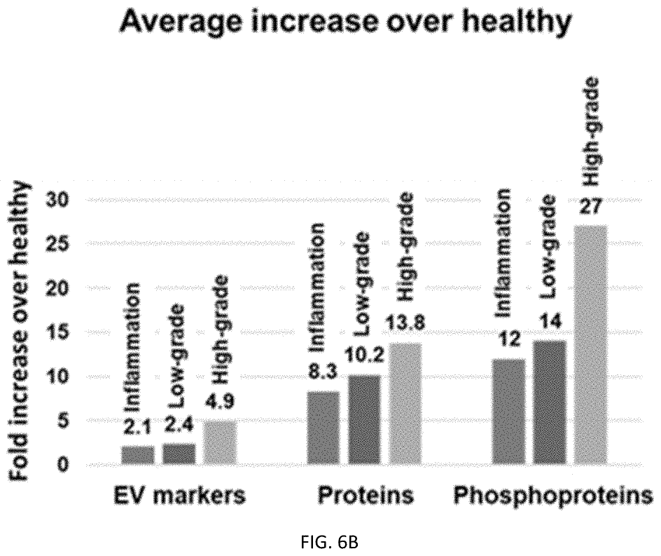

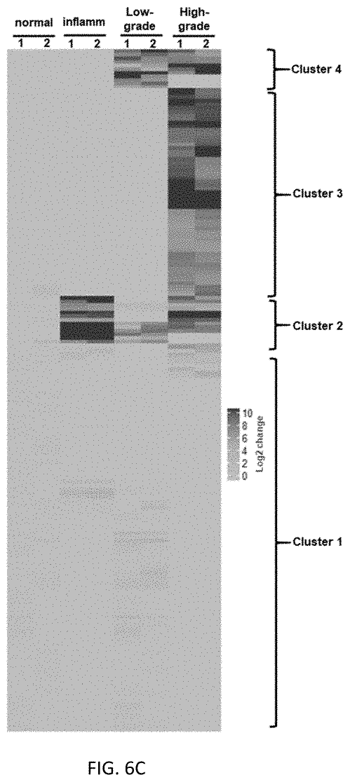

For the first experiment this disclosure pooled together 27 healthy, 10 inflammation, 4 low-grade and 21 high-grade bladder cancer urine samples into 4 separate samples analyzed in triplicate. In this large pooled experiment, 259 proteins and 222 phosphoproteins were quantified as significantly increased (>4-fold) in bladder cancer. The statistically significant protein and phosphorite changes were identified by P-value as significant based on a two-sample t-test with a permutation-based 0.05 FDR cutoff. The overall quantitation numbers, EV markers, EV protein and phosphoprotein intensity differences and select phosphoproteins log-scale intensity heatmap are shown in FIGS. 6A-6C. As denoted on the heatmap, this disclosure was able to group together select proteins and phosphoproteins into clusters that can effectively differentiate low-grade and high-grade bladder cancer from healthy control samples or patients with other non-cancerous bladder indications. It will be understood by one skilled in the art that other relevant conditions for an EV biomarker can be used as control samples.

This disclosure then carried out the follow-up EVtrap+LCMS experiments using individual urine samples. For the phosphoproteome analysis, this disclosure processed 23 healthy urine samples, 4 inflammation/infection samples and 18 bladder cancer samples. For the proteome evaluation, the distribution was 23 healthy, 4 inflammation, and 11 bladder cancer samples. In total, each individual sample was analyzed in triplicate by quantitative mass spectrometry, producing a total of 2,769 quantified unique proteins and over 11,000 quantified unique phosphorites. This disclosure carried out linear regression statistical analysis at P-value <0.05 to narrow down the panel of potential biomarkers to those with highest statistical significance and largest intensity changes (majority were >10-fold increased).

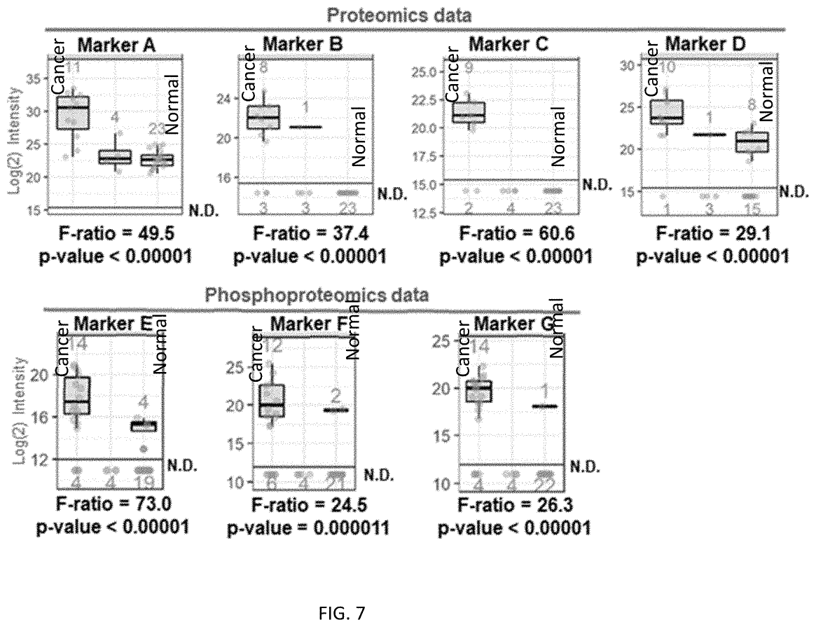

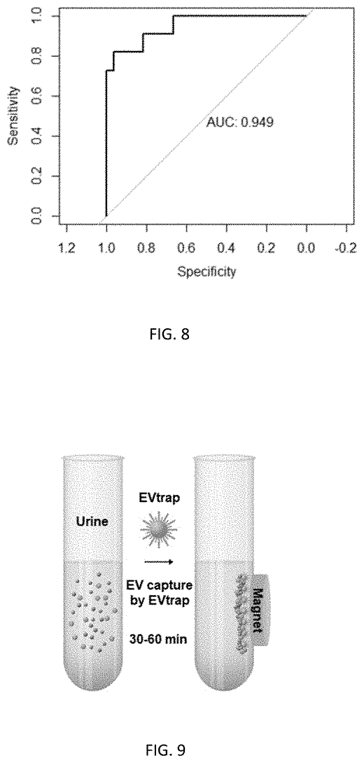

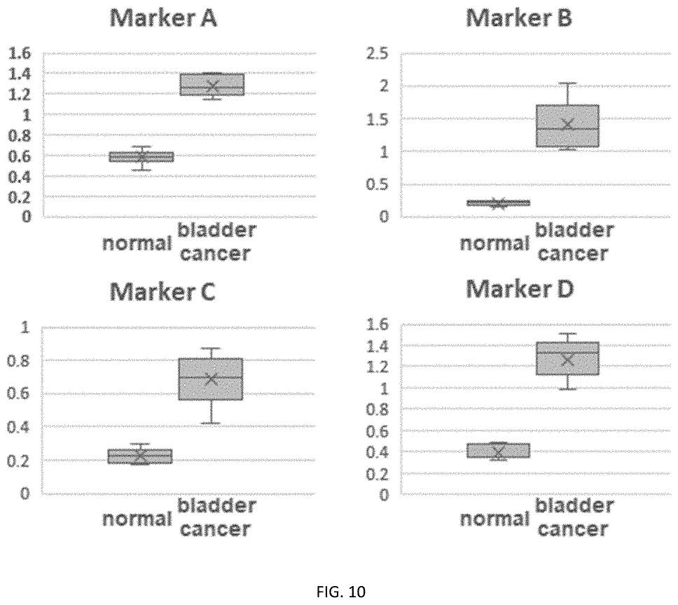

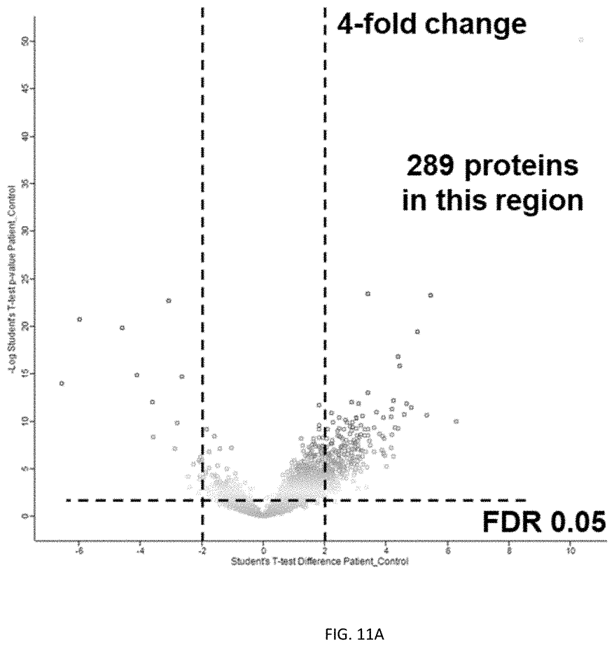

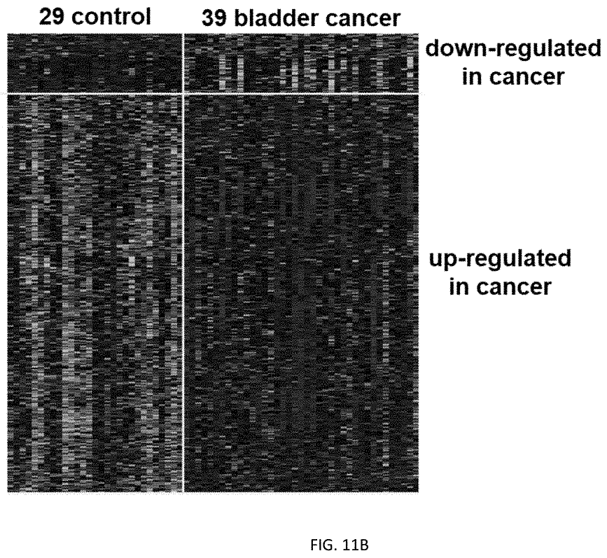

FIG. 7 shows the box-and-whisker plot examples of a few of the select potential biomarkers. The scale is set at log intensity for easy interpretation, so the average difference in signal for most is 10-70× increase in bladder cancer compared to the two control groups. For most of these markers, the signal was not detectable in the majority of the control samples. The F-ratio values and P-values are included under each plot. Both proteomics and phosphoproteomics datasets were effective at finding differentiated markers, although phosphoproteomics was more likely to find unique bladder cancer proteins that were present at very low amounts.

From these data, this disclosure generated an initial list containing 83 proteins and phosphoproteins that were consistently present in bladder cancer urine but at very low amounts or not detectable in any control samples. We carried out additional LC-MS analyses of 29 new healthy urine EV samples and 39 new bladder cancer urine EV samples. These efforts identified over 7,000 proteins and over 5,000 phosphoproteins. Finally, the panel of potential biomarkers from all of the above experiments contained 594 proteins and phosphoproteins that have the potential to differentiate bladder cancer from non-cancer urine samples.

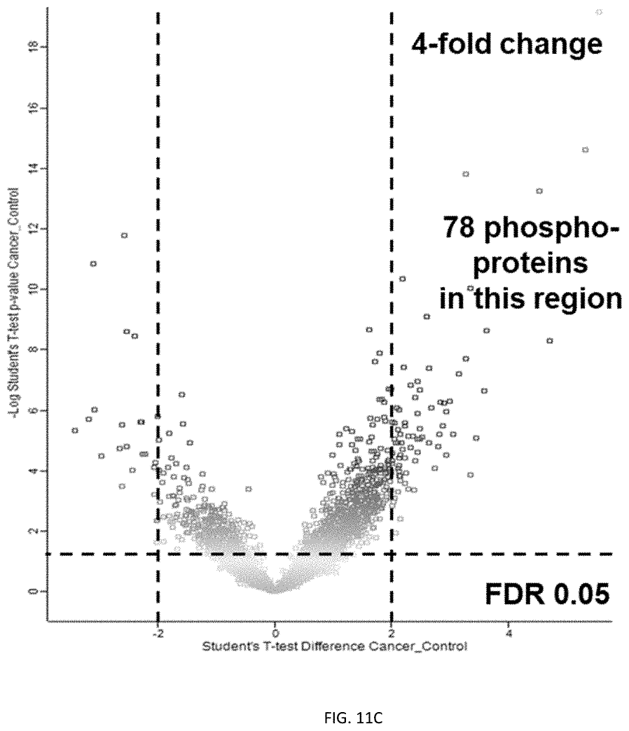

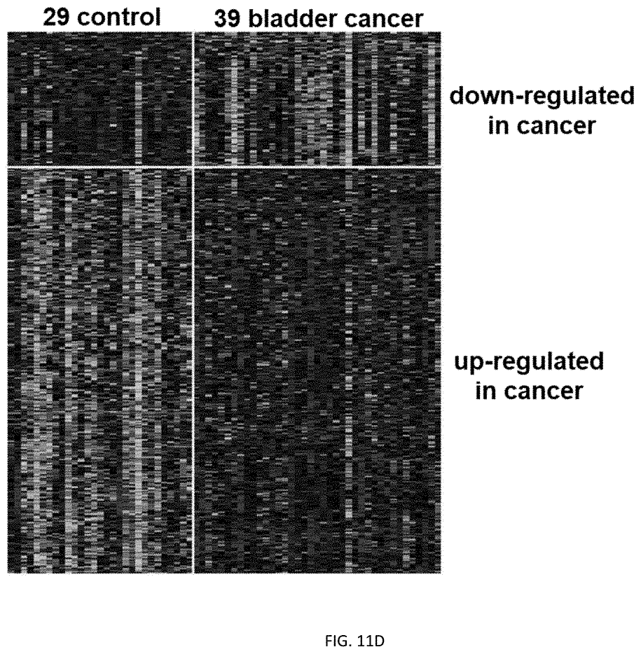

We identified 289 upregulated proteins and 78 upregulated phosphoproteins that increased in abundance at least 4-fold in bladder cancer at P-value <0.05. These results are visualized in FIG. 11A with the volcano plot, and FIG. 11B with the heatmap of the EV proteins that were significantly up- or downregulated in bladder cancer compared to healthy controls. Likewise, FIG. 11C shows the volcano plot, and FIG. 11D the heatmap of the EV phosphoproteins that were significantly up- or downregulated in bladder cancer compared to healthy controls.

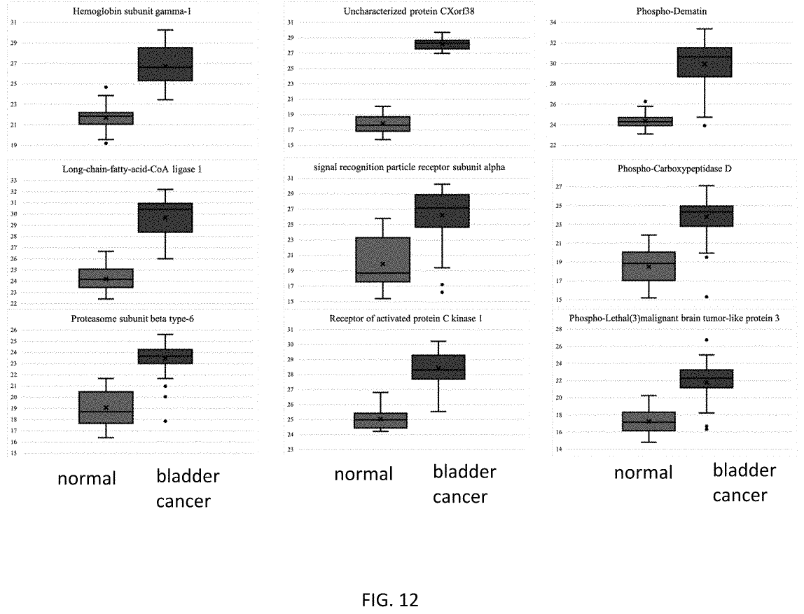

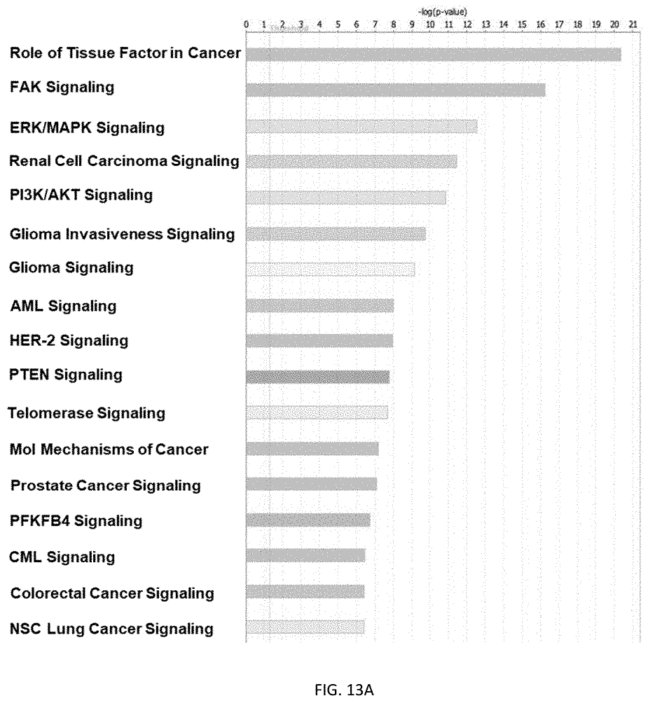

From this dataset, in FIG. 12 we selected several proteins and phosphoproteins to create box-and-whisker plots for quantitative LC-MS data of markers capable of differentiating bladder cancer urine from healthy controls (normal urine n=29; bladder cancer urine n=39). The scale is set at log intensity for easy interpretation, so the average difference in signal for most is at least 10× increase in bladder cancer compared to the control group.

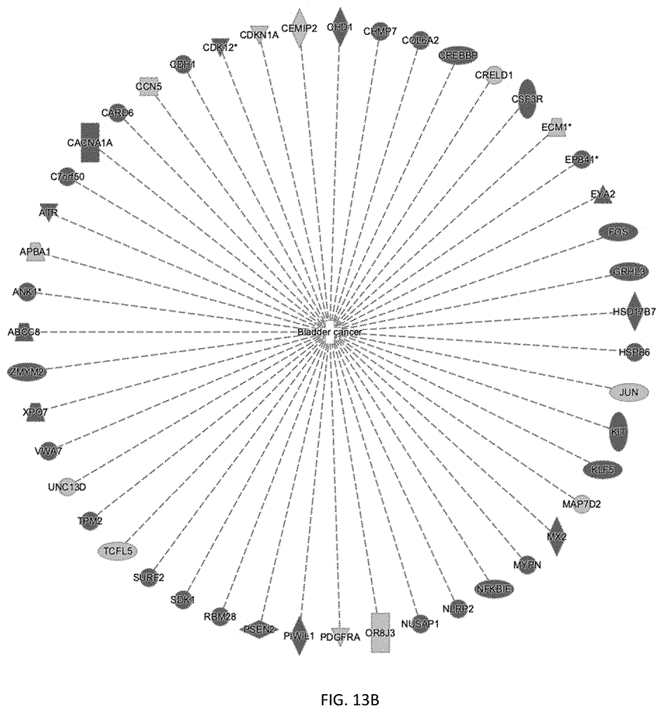

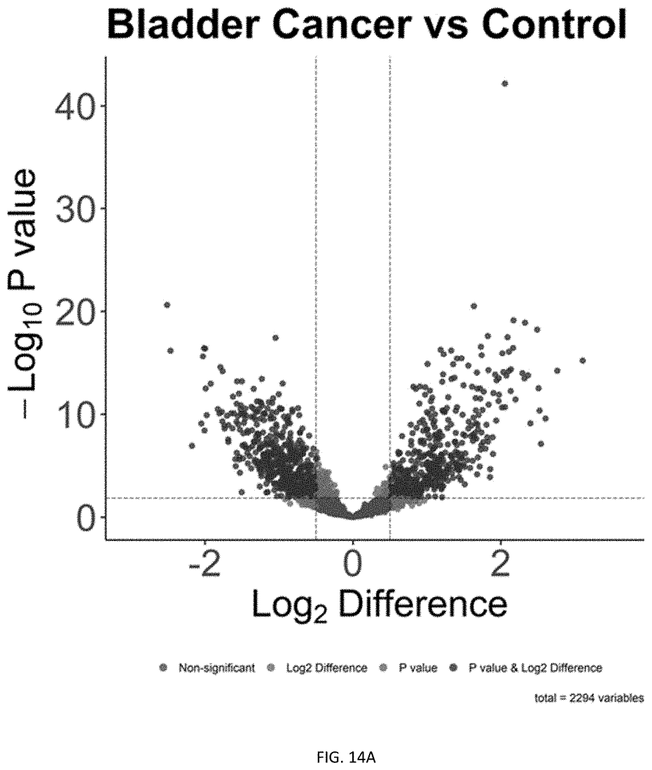

Analysis of Gene Ontology pathways of these data showed a significant enrichment of cancer networks in upregulated proteins and phosphoproteins (FIG. 13A). Bladder cancer was one of those significantly enriched networks, with hundreds of proteins correlated with bladder cancer (FIG. 13B). This further underscores that urinary exosomes have a unique ability to serve as a surrogate for bladder tissue samples, as many known bladder cancer-related markers are also upregulated in urine EVs. In addition, we found several significantly upregulated kinases and kinase pathways, further underscoring the importance of protein phosphorylation in bladder cancer development and progression.

Finally, we carried out a validation experiment for bladder cancer biomarkers using a new cohort of 74 control urine samples and 102 urine samples from bladder cancer individuals. As before, the EVs from urine were enriched using EVtrap, and the resulting EV proteins were identified and quantified using LC-MS. This validation experiment confirmed many of the bladder cancer EV protein biomarkers previously identified.

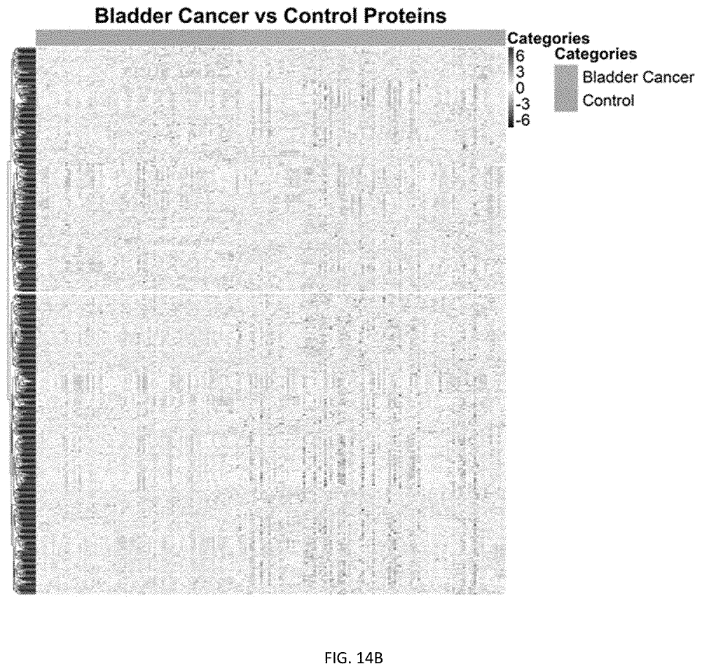

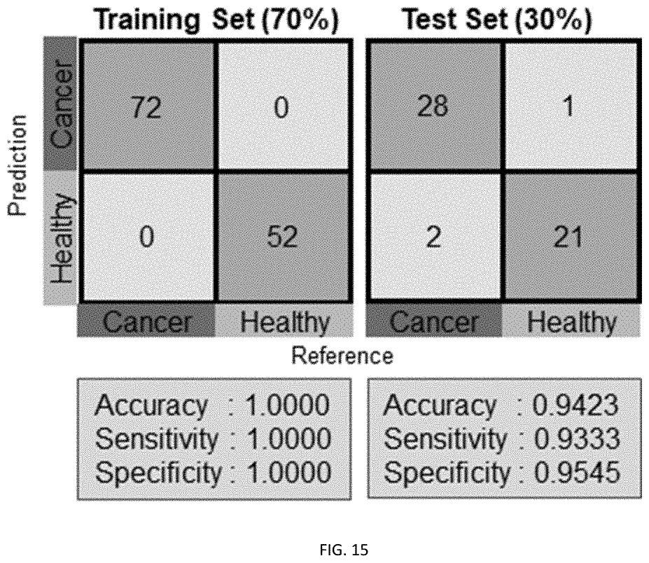

The most recent results for the significantly upregulated and downregulated EV proteins in bladder cancer are visualized in FIG. 14A with the volcano plot and FIG. 15B with the heatmap.

### Example 2: Bladder Cancer Biomarkers Panel for Diagnosis by Urine Test

The samples from Example 1 were subsequently split into a training set (70% of the samples) and test set (30%). The algorithm was trained using the training set, and then checked on the test set to see its accuracy. As shown in FIG. 15, the accuracy for bladder cancer detection in the training set was the perfect 1, and for the test set an outstanding 0.94.

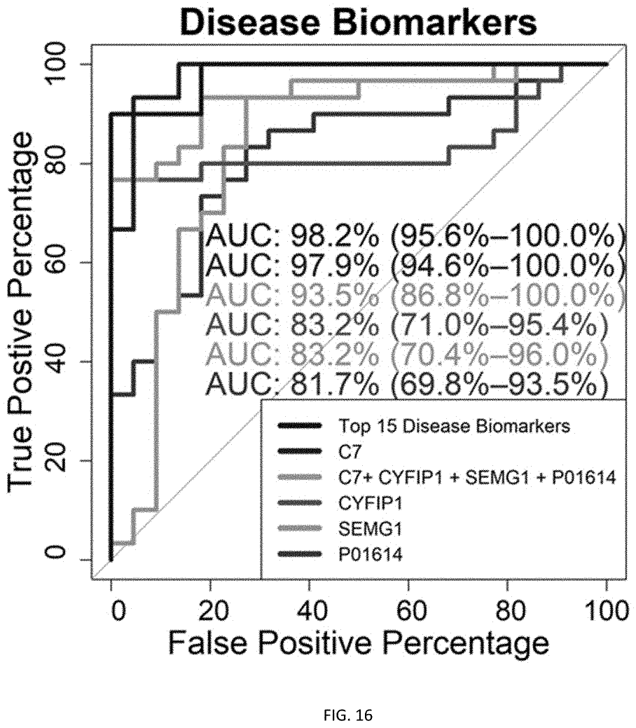

We also did ROC curve analysis of the top markers validated in this experiment, and found that the best combination of markers can result in AUC of 98% (FIG. 16). Overall, these data confirm the ability of using the novel urine EV markers discovered by the method described in Example 1 and listed in Table Ito successfully differentiate bladder cancer samples from non-cancer controls. Table I lists unique proteins and phosphoproteins capable of differentiating bladder cancer urine from healthy urine and inflammation control urine.

TABLE I
Biomarkers (proteins and phosphoproteins) capable of differentiating bladder
cancer urine from healthy urine and inflammation control urine

| | | | | | |
|---|---|---|---|---|---|
| CYFIP1 | SEMG1 | IGKV2D-40 | PRPS1 | IGKV1-17 | PDIA5 |
| EMILIN2 | IGKC | EPX | EIF2S1 | PSME1 | YKT6 |
| IRF2BPL | PON1 | AXL | PLG | ANK1 | SF3A3 |
| FGB | FUS | CEACAM8 | F12 | HTT | NAAA |
| APBB1IP | RPS19 | GK | A2M | TFPI2 | PSD3 |
| PTPRC | PSMD8 | FRK | C3 | MARCKSL1 | PRG4 |
| CFH | VASP | EIF1AX | C5 | PXN | HNF4A |
| VIM | PLTP | S100A10 | APOA1 | BASP1 | GPX2 |
| MSN | SNRPE | ARHGAP1 | APOC1 | AMPD3 | GBE1 |
| ARHGAP4 | EWSR1 | EPS8 | KLKB1 | HSP90AA2P | GIT2 |
| NUMA1 | SRSF1 | DDX39B | HRG | SF3A1 | BOLA3 |
| PDCD4 | ILF3 | CTTN | PROS1 | RASAL3 | RRM2B |
| LRRK2 | APOF | EIF4H | C8A | REPS2 | CPD |
| NEK9 | HABP2 | MAPK14 | HNRNPC | ATXN2L | RPS27 |
| MED9 | NONO | CDC37 | C7 | HSPH1 | L3MBTL3 |
| SAA2-SAA4 | SF1 | OCC1 | C6 | KCTD12 | TMEM163 |
| IGHV2-26 | MAPRE1 | S100A13 | CPN1 | PNN | ASPSCR1 |
| ZNF233 | ZYX | C14orf166 | VCL | RTN4 | UBE2Z |
| APOA2 | CPSF6 | PSMD9 | ITIH2 | NUDC | PEAK3 |
| APOC4-APOC2 | HP1BP3 | ZNF207 | ITIH1 | HBG1 | EPHA1 |
| APOL1 | FKBP15 | GMFG | TKT | ACSL1 | PATL1 |
| HMGB3 | SLC25A24 | C2 | STIP1 | CXorf38 | RAN |
| FCN3 | DNMBP | RPL7 | HDGF | SRPRA | OTUD5 |
| F13A1 | SND1 | WARS | S100A12 | CARM1 | LARP7 |
| HPR | FUBP1 | EIF4B | LMNB2 | PSMB6 | UBASH3B |
| C1QA | BIN2 | APEX1 | FAM49B | RACK1 | ARAP1 |
| C1QB | SEPT9 | CFP | TLN1 | CCSER1 | DDX1 |
| C1QC | HAPB2 | ADSS | WIPF1 | UBXN11 | SEPT6 |
| FN1 | MAP2K2 | SUB1 | MECP2 | ZNF613 | TRIM24 |
| MMP1 | TJP2 | PAFAH1B2 | LCP2 | TMEM43 | TLE4 |
| C4BPA | TPD52L2 | SRSF3 | SRSF6 | CTXN1 | FLII |
| APOB | BUB3 | ITIH3 | IQCB1 | BCAP31 | WDR33 |
| SERPIND1 | BCAS1 | BAX | RCSD1 | PSMB9 | SPTA1 |
| C8B | KRAS | AP1B1 | CHRDL2 | SERPINB9 | EP300 |
| C8G | FGA | CBX3 | PRAM1 | H2AC21 | ZFYVE20 |
| PLEK | FGG | PSMD6 | SRRM2 | KIFAP3 | CTNND1 |
| NCF1 | S100A9 | TGFBI | INPP5D | CLPB | L1CAM |
| STMN1 | HSP90AB1 | SRSF7 | SLC26A4 | LRMP | SRSF10 |
| ORM2 | MMP2 | FERMT3 | NCF1B | MCMBP | MAP2K3 |
| NFKB1 | TACSTD2 | CNN2 | KDM1A | GSDMD | C1orf35 |
| NCF2 | CDH1 | CFHR5 | STK17B | PTGES2 | NFX1 |
| EIF2S2 | SRC | APMAP | NADK | RPS10 | PMS2 |
| LMNB1 | YES1 | PADI4 | OXSR1 | TRIM9 | IPCEF1 |
| C4BPB | EPCAM | PA2G4 | SLC4A1 | LBR | JUP |
| FLNA | S100A1 | FLNB | SLC2A3 | RPL32 | SIRT2 |
| CBL | MAPK1 | APOM | TOP2A | LDHC | ACKR3 |
| S100A4 | MARCKS | HP | TRP | DIAPH3 | ADAR |
| IPO11 | ADGRG6 | LPA | COL1A1 | LYAR | SUPT5H |
| NFASC | PYCR3 | KPNB1 | HAPLN3 | DCPS | LSM4 |
| CUL4A | CHAMP1 | ACAA1 | MYH13 | CUL5 | HNRNPM |
| SEC62 | GMDS | TGM2 | NDUFB11 | TRIOBP | EIF4G2 |
| RPS14 | FARSA | UAP1L1 | HK2 | EIF4A3 | SF3B2 |
| ATP5IF1 | TMEM33 | SLC6A13 | ABHD3 | HAO2 | DHX57 |
| SCAMP4 | POLR1B | SEPTIN6 | ANGPTL4 | RCC2 | STARD10 |
| HNRNPUL2 | DDX46 | LDLR | AKR1C2 | CBX5 | ATN1 |
| TNFAIP8 | MRPL58 | EHD2 | RNASE3 | HBD | SRF |
| TMX1 | MTM1 | EPB42 | IDI1 | ALDH3A2 | BDP1 |
| ATG3 | CNOT1 | DEK | SEC24C | PLOD3 | NOLC1 |
| EPB41L3 | MLKL | DNAJC17 | SEC23B | TSNAX | CASP9 |
| ACOX1 | NECTIN1 | EXOC3 | DDX17 | NCL | NFIA |
| SUCLG1 | CUL2 | PSTPIP1 | DEPDC1B | ABCC11 | SMC3 |
| PSPC1 | GAR1 | RPL10 | PCDHA3 | WDR5 | FLVCR1 |
| MCM5 | DDI2 | FAM98A | EGFLAM | TM9SF2 | RP2 |
| VAPA | AIMP1 | RIC8A | MYO15B | EFCAB13 | PRPF38B |
| RAB2B | SRP14 | CIB1 | RELCH | TBC1D24 | MCM3 |
| RBBP4 | BSG | MTREX | SNX27 | DIAPH1 | ARHGAP9 |
| SAA4 | DMTN | NCKAP1L | IRF4 | HSD17B2 | UBAP2 |
| G3BP1 | CORO1B | PRMT1 | ENAH | CCDC124 | BRD9 |
| TENM1 | SKP1 | SF3A2 | BZW1 | GATA5 | HNRNPA0 |
| SLC4A11 | ZBTB5 | CHID1 | CSTF3 | CTSO | KMT2C |
| PRDX3 | DDX23 | CRAT | SNRPB2 | WDR13 | NRG1 |
| RPL28 | TMEM41B | PLEKHA6 | ATP2B4 | PSMF1 | GLYR1 |
| HMGCR | BLOC1S1 | WAS | MYO5C | HBA1 | MAPRE3 |
| LACTB | SELENOS | GIGYF2 | BRI3 | DEFB1 | SNRPC |
| TRBC2 | PUS1 | CAMP | H2BC14 | TTC21B | LTB4R |
| PPP6C | DNAH9 | GMIP | SPAG17 | GAPVD1 | LRWD1 |
| SCARF2 | SCG3 | PURB | PDE9A | TNKS1BP1 | MYLK3 |
| ADSL | DYNC2H1 | UBTF | XPO4 | VAV1 | RANBP10 |
| SNRPA1 | TUBB2A | SNRPD2 | PFAS | GALNT18 | PRPF19 |
| RPL22L1 | HMGB2 | SNRPGP15 | ZNF268 | KRT82 | ZFR |
| PPM1A | NOP56 | DNAH14 | PARP14 | GALNT16 | DDX42 |
| ABCE1 | PRELP | PRG3 | NBEAL2 | TPP2 | TM9SF3 |
| SMU1 | NOP58 | PSMA1 | PSMD4 | PGM2L1 | H6PD |
| FMC1 | XPO1 | ARHGEF2 | NRBP1 | CCDC22 | PKN1 |
| ARHGAP27 | APOC4 | POSTN | IMPDH1 | KIF13B | SPN |
| HBG2 | B2M | CSE1L | PRKDC | HBB | VWA7 |
| EIF3M | ROCK2 | TCEAL9 | APOC2 | ADSS2 | PRSS2 |
| SLC25A13 | SRRM1 | TMF1 | FARSB | CD5L | CD47 |
| RIPOR2 | LMLN | MAP4 | THBS2 | CARS1 | SH3KBP1 |
| PRDM7 | TRA2B | ATXN10 | MPP1 | PREP | TMA7 |
| LY75 | UMPS | RAC2 | HDGFL2 | DNM1L | CD5L |
| FTSJ1 | ZNF276 | ILF2 | SH3BP1 | MACF1 | COX7C |
| RRAS2 | HINT3 | CHST3 | IL18 | SPINT1 | GNG2 |
| PPA2 | VNN1 | YOD1 | SLC47A1 | NPHS2 | CEACAM5 |
| AQP1 | TMEM27 | SMR3B | ENTPD6 | CA12 | MASP1 |
| DNASE1L1 | GAS1 | ACADVL | AK3 | IDE | HMOX1 |
| DBT | CAMK2D | IQCC | CD109 | EGF | SERPINA10 |
| DNAJB2 | CYSTM1 | MAN1B1 | TSTA3 | PPP1R12A | PPBP |
| MUC20 | ARL6IP5 | NEBL | C21orf33 | TMEM106B | SEMA3C |

In this disclosure, the applicants have demonstrated the feasibility and clinical ability of the newly discovered urine biomarkers to discriminate between cancer and non-cancer samples. This disclosure discovered hundreds of proteins and phosphoproteins that appear to change significantly in bladder cancer urine EVs compared to healthy or inflamed urine samples. This disclosure narrowed this list down to about 590 potential markers that demonstrate the most statistically significant differentiation between the cases. Most of them show at least 4-fold change on a highly consistent and reproducible level at p-value <0.0001.

This highlights the primary advantage of using phosphoproteomic analysis for biomarker discovery. It is not necessarily the case that these proteins are always differentially phosphorylated in urine EVs. But rather that they are very low-abundant signaling proteins which are loaded into EVs and are commonly phosphorylated. Typically, they are not detectable by standard proteomic analysis. Phosphoproteome enrichment thus removes the common higher abundant proteins and peptides, while isolating and bringing to the forefront these phosphorylated low-abundant targets. The ability to increase the volume of urine utilized for phosphoproteomics from 0.2 mL to about 10 mL also contributes to the detection and quantitation of these targets. Therefore, even though multiple biomarkers were discovered through phosphoproteomic experiments, it is most likely the whole protein amount changes in the EVs. And, thus, these targets can be detectable by regular antibody assays without having to develop specific phospho-antibodies.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

### REFERENCES

1. Nagrath, S., Sequist, L. V., Maheswaran, S., Bell, D. W., lrimia, D., Ulkus, L., Smith, M. R., Kwak, E. L., Digumarthy, S., Muzikansky, A., Ryan, P., Balis, U. J., Tompkins, R. G., Haber, D. A., and Toner, M. (2007) Isolation of rare circulating tumour cells in cancer patients by microchip technology. Nature 450,1235-1239.
2. Flaig, T. W., Wilson, S., van Bokhoven, A., Varella-Garcia, M., Wolfe, P., Maroni, P., Genova, E. E., Morales, D., and Lucia, M. S. (2011) Detection of circulating tumor cells in metastatic and clinically localized urothelial carcinoma. Urology 78, 863-867.
3. Harel, M., Oren-Giladi, P., Kaidar-Person, O., Shaked, Y., and Geiger, T. (2015) Proteomics of microparticles with SILAC Quantification (PROMIS-Quan): a novel proteomic method for plasma biomarker quantification. Mol Cell Proteomics 14, 1127-1136.
4. Milane, L., Singh, A., Mattheolabakis, G., Suresh, M., and Amiji, M. M. (2015) Exosome mediated communication within the tumor microenvironment. J Control Release 219, 278-294.
5. Cocucci, E., and Meldolesi, J. (2015) Ectosomes and exosomes: shedding the confusion between extracellular vesicles. Trends in cell biology 25, 364-372.
6. Lin, J., Li, J., Huang, B., Liu, J., Chen, X., Chen, X. M., Xu, Y. M., Huang, L. F., and Wang, X. Z. (2015) Exosomes: novel biomarkers for clinical diagnosis. TheScientificWorldJournal 2015, 657086.
7. Melo, S. A., Luecke, L. B., Kahlert, C., Fernandez, A. F., Gammon, S. T., Kaye, J., LeBleu, V. S., Mittendorf, E. A., Weitz, J., Rahbari, N., Reissfelder, C., Pilarsky, C., Fraga, M. F., Piwnica-Worms, D., and Kalluri, R. (2015) Glypican-1 identifies cancer exosomes and detects early pancreatic cancer. Nature 523, 177-182.
8. Ridder, K., Sevko, A., Heide, J., Dams, M., Rupp, A. K., Macas, J., Starmann, J., Tjwa, M., Plate, K. H., Sultmann, H., Altevogt, P., Umansky, V., and Momma, S. (2015) Extracellular vesicle-mediated transfer of functional RNA in the tumor microenvironment. Oncoimmunology 4, e1008371.
9. Dobrowolski, R., and De Robertis, E. M. (2012) Endocytic control of growth factor signalling: multivesicular bodies as signalling organelles. Nat RevMol Cell Biol 13, 53-60.
10. Costa-Silva, B., Aiello, N. M., Ocean, A. J., Singh, S., Zhang, H., Thakur, B. K., Becker, A., Hoshino, A., Mark, M. T., Molina, H., Xiang, J., Zhang, T., Theilen, T. M., Garcia-Santos, G., Williams, C., Ararso, Y., Huang, Y., Rodrigues, G., Shen, T. L., Labori, K. J., Lothe, I. M., Kure, E. H., Hernandez, J., Doussot, A., Ebbesen, S. H., Grandgenett, P. M., Hollingsworth, M. A., Jain, M., Mallya, K., Batra, S. K., Jarnagin, W. R., Schwartz, R. E., Matei, I., Peinado, H., Stanger, B. Z., Bromberg, J., and Lyden, D. (2015) Pancreatic cancer exosomes initiate pre-metastatic niche formation in the liver. Nature cell biology 17, 816-826.
11. An, T., Qin, S., Xu, Y., Tang, Y., Huang, Y., Situ, B., Inal, J. M., and Zheng, L. (2015) Exosomes serve as tumour markers for personalized diagnostics owing to their important role in cancer metastasis. Journal of extracellular vesicles 4, 27522.
12. Sokolova, V., Ludwig, A. K., Hornung, S., Rotan, O., Horn, P. A., Epple, M., and Giebel, B. (2011) Characterisation of exosomes derived from human cells by nanoparticle tracking analysis and scanning electron microscopy. Colloids and surfaces. B, Biointerfaces 87, 146-150.
13. Boukouris, S., and Mathivanan, S. (2015) Exosomes in bodily fluids are a highly stable resource of disease biomarkers. Proteomics Clin Appl 9, 358-367.
14. van der Mijn, J. C., Sol, N., Mellema, W., Jimenez, C. R., Piersma, S. R., Dekker, H., Schutte, L. M., Smit, E. F., Broxterman, H. J., Skog, J., Tannous, B. A., Wurdinger, T., and Verheul, H. M. (2014) Analysis of AKT and ERK1/2 protein kinases in extracellular vesicles isolated from blood of patients with cancer. Journal of extracellular vesicles 3, 25657.
15. Berrade, L., Garcia, A. E., and Camarero, J. A. (2011) Protein microarrays: novel developments and applications. Pharm Res 28, 1480-1499.
16. Oberprieler, N. G., and Tasken, K. (2011) Analysing phosphorylation-based signalling networks by phospho flow cytometry. Cellular signalling 23, 14-18.
17. Gonzales, P. A., Pisitkun, T., Hoffert, J. D., Tchapyjnikov, D., Star, R. A., Kleta, R., Wang, N. S., and Knepper, M. A. (2009) Large-scale proteomics and phosphoproteomics of urinary exosomes. Journal of the American Society of Nephrology: JASN 20, 363-379.
18. Palmisano, G., Jensen, S. S., Le Bihan, M. C., Laine, J., McGuire, J. N., Pociot, F., and Larsen, M. R. (2012) Characterization of membrane-shed microvesicles from cytokine-stimulated beta-cells using proteomics strategies. Mol Cell Proteomics 11, 230-243.
19. Chen, I. H., Xue, L., Hsu, C. C., Paez, J. S., Pan, L., Andaluz, H., Wendt, M. K., lliuk, A. B., Zhu, J. K., and Tao, W. A. (2017) Phosphoproteins in extracellular vesicles as candidate markers for breast cancer. Proc Natl Acad Sci USA 114, 3175-3180.
20. Wu, X., Li, L., Iliuk, A., and Tao, W. A. (2018) Highly Efficient Phosphoproteome Capture and Analysis from Urinary Extracellular Vesicles. J Proteome Res 17, 3308-3316.
21. Lotvall, J., Hill, A. F., Hochberg, F., Buzas, E. I., Di Vizio, D., Gardiner, C., Gho, Y. S., Kurochkin, I. V., Mathivanan, S., Quesenberry, P., Sahoo, S., Tahara, H., Wauben, M. H., Witwer, K. W., and Thery, C. (2014) Minimal experimental requirements for definition of extracellular vesicles and their functions: a position statement from the International Society for Extracellular Vesicles. Journal of extracellular vesicles 3, 26913.
22. Witwer, K. W., Buzas, E. I., Bemis, L. T., Bora, A., Lasser, C., Lotvall, J., Nolte-′t Hoen, E. N., Piper, M. G., Sivaraman, S., Skog, J., Thery, C., Wauben, M. H., and Hochberg, F. (2013) Standardization of sample collection, isolation and analysis methods in extracellular vesicle research. Journal of extracellular vesicles 2, 1-25.
23. Lamparski, H. G., Metha-Damani, A., Yao, J. Y., Patel, S., Hsu, D. H., Ruegg, C., and Le Pecq, J. B. (2002) Production and characterization of clinical grade exosomes derived from dendritic cells. Journal of immunological methods 270, 211-226.
24. Nakai, W., Yoshida, T., Diez, D., Miyatake, Y., Nishibu, T., Imawaka, N., Naruse, K., Sadamura, Y., and Hanayama, R. (2016) A novel affinity-based method for the isolation of highly purified extracellular vesicles. Sci Rep 6, 33935.
25. Stranska, R., Gysbrechts, L., Wouters, J., Vermeersch, P., Bloch, K., Dierickx, D., Andrei, G., and Snoeck, R. (2018) Comparison of membrane affinity-based method with size-exclusion chromatography for isolation of exosome-like vesicles from human plasma. J Transl Med 16, 1.
26. Zeringer, E., Li, M., Barta, T., Schageman, J., Pedersen, K. W., Neurauter, A., Magdaleno, S., Setterquist, R., and Vlassov, A. V. (2013) Methods for the extraction and RNA profiling of exosomes. World J Methodol 3, 11-18.
27. Niu, Z., Pang, R. T. K., Liu, W., Li, Q., Cheng, R., and Yeung, W. S. B. (2017) Polymer-based precipitation preserves biological activities of extracellular vesicles from an endometrial cell line. PLoS One 12, e0186534.
28. Mathivanan, S., Lim, J. W., Tauro, B. J., Ji, H., Moritz, R. L., and Simpson, R. J. (2010) Proteomics analysis of A33 immunoaffinity-purified exosomes released from the human colon tumor cell line LIM1215 reveals a tissue-specific protein signature. Mol Cell Proteomics 9, 197-208.
29. Yoo, C. E., Kim, G., Kim, M., Park, D., Kang, H. J., Lee, M., and Huh, N. (2012) A direct extraction method for microRNAs from exosomes captured by immunoaffinity beads. Anal Biochem 431, 96-98.
30. Enderle, D., Spiel, A., Coticchia, C. M., Berghoff, E., Mueller, R., Schlumpberger, M., Sprenger-Haussels, M., Shaffer, J. M., Lader, E., Skog, J., and Noerholm, M. (2015) Characterization of RNA from Exosomes and Other Extracellular Vesicles Isolated by a Novel Spin Column-Based Method. PLoS One 10, e0136133.
31. Welton, J. L., Webber, J. P., Botos, L. A., Jones, M., and Clayton, A. (2015) Ready-made chromatography columns for extracellular vesicle isolation from plasma. Journal of extracellular vesicles 4, 27269.
32. Tang, Y. T., Huang, Y. Y., Zheng, L., Qin, S. H., Xu, X. P., An, T. X., Xu, Y., Wu, Y. S., Hu, X. M., Ping, B. H., and Wang, Q. (2017) Comparison of isolation methods of exosomes and exosomal RNA from cell culture medium and serum. Int J Mol Med 40, 834-844.
33. Bijnsdorp, I. V., Maxouri, O., Kardar, A., Schelfhorst, T., Piersma, S. R., Pham, T. V., Vis, A., van Moorselaar, R. J., and Jimenez, C. R. (2017) Feasibility of urinary extracellular vesicle proteome profiling using a robust and simple, clinically applicable isolation method. Journal of extracellular vesicles 6, 1313091.
34. Royo, F., Zuniga-Garcia, P., Sanchez-Mosquera, P., Egia, A., Perez, A., Loizaga, A., Arceo, R., Lacasa, I., Rabade, A., Arrieta, E., Bilbao, R., Unda, M., Carracedo, A., and Falcon-Perez, J. M. (2016) Different EV enrichment methods suitable for clinical settings yield different subpopulations of urinary extracellular vesicles from human samples. Journal of extracellular vesicles 5, 29497.
35. Liang, L. G., Sheng, Y. F., Zhou, S., Ind, F., Li, L., Demirci, U., and Wang, S. (2017) An Integrated Double-Filtration Microfluidic Device for Detection of Extracellular Vesicles from Urine for Bladder Cancer Diagnosis. Methods Mol Biol 1660, 355-364.
36. Van Deun, J., Mestdagh, P., Sormunen, R., Cocquyt, V., Vermaelen, K., Vandesompele, J., Bracke, M., De Wever, O., and Hendrix, A. (2014) The impact of disparate isolation methods for extracellular vesicles on downstream RNA profiling. Journal of extracellular vesicles 3, 1-14.
37. Taylor, D. D., and Shah, S. (2015) Methods of isolating extracellular vesicles impact down-stream analyses of their cargoes. Methods 87, 3-10.
38. Cvjetkovic, A., Lotvall, J., and Lasser, C. (2014) The influence of rotor type and centrifugation time on the yield and purity of extracellular vesicles. Journal of extracellular vesicles 3, 1-11.
39. Keerthikumar, S., Chisanga, D., Ariyaratne, D., Al Saffar, H., Anand, S., Zhao, K., Samuel, M., Pathan, M., Jois, M., Chilamkurti, N., Gangoda, L., and Mathivanan, S. (2016) ExoCarta: A Web-Based Compendium of Exosomal Cargo. J Mol Biol 428, 688-692.
40. Mathivanan, S., Fahner, C. J., Reid, G. E., and Simpson, R. J. (2012) ExoCarta 2012: database of exosomal proteins, RNA and lipids. Nucleic Acids Res 40, D1241-1244.
41. Simpson, R. J., Kalra, H., and Mathivanan, S. (2012) ExoCarta as a resource for exosomal research. Journal of extracellular vesicles 1, 1-6.
42. Wang, T., Anderson, K. W., and Turko, I. V. (2017) Assessment of Extracellular Vesicles Purity Using Proteomic Standards. Anal Chem 89, 11070-11075.
43. Jayachandran, M., Miller, V. M., Heit, J. A., and Owen, W. G. (2012) Methodology for isolation, identification and characterization of microvesicles in peripheral blood. Journal of immunological methods 375, 207-214.
44. Macey, M., Azam, U., McCarthy, D., Webb, L., Chapman, E. S., Okrongly, D., Zelmanovic, D., and Newland, A. (2002) Evaluation of the anticoagulants EDTA and citrate, theophylline, adenosine, and dipyridamole (CTAD) for assessing platelet activation on the ADVIA 120 hematology system. Clinical chemistry 48, 891-899.
45. Gyorgy, B., Paloczi, K., Kovacs, A., Barabas, E., Beko, G., Varnai, K., Pallinger, E., Szabo-Taylor, K., Szabo, T. G., Kiss, A. A., Falus, A., and Buzas, E. I. (2014) Improved circulating microparticle analysis in acid-citrate dextrose (ACD) anticoagulant tube. Thrombosis research 133, 285-292.
46. Gholizadeh, S., Shehata Draz, M., Zarghooni, M., Sanati-Nezhad, A., Ghavami, S., Shafiee, H., and Akbari, M. (2017) Microfluidic approaches for isolation, detection, and characterization of extracellular vesicles: Current status and future directions. Biosens Bioelectron 91, 588-605.
47. Xu, R., Greening, D. W., Zhu, H. J., Takahashi, N., and Simpson, R. J. (2016) Extracellular vesicle isolation and characterization: toward clinical application. The Journal of clinical investigation 126, 1152-1162.
48. Galan, J. A., Guo, M., Sanchez, E. E., Cantu, E., Rodriguez-Acosta, A., Perez, J. C., and Tao, W. A. (2008) Quantitative analysis of snake venoms using soluble polymer-based isotope labeling. Mol Cell Proteomics 7, 785-799.
49. Iliuk, A. B., Martin, V. A., Alicie, B. M., Geahlen, R. L., and Tao, W. A. (2010) In-depth analyses of kinase-dependent tyrosine phosphoproteomes based on metal ion functionalized soluble nanopolymers. Mol Cell Proteomics 9, 2162-2172.
50. Guo, M., Galan, J., and Tao, W. A. (2007) Soluble nanopolymer-based phosphoproteomics for studying protein phosphatase. Methods 42, 289-297.
51. Zhou, F., Galan, J., Geahlen, R. L., and Tao, W. A. (2007) A novel quantitative proteomics strategy to study phosphorylation-dependent peptide-protein interactions. J Proteome Res 6, 133-140.
52. Wang, L., Yang, L., Pan, L., Kadasala, N. R., Xue, L., Schuster, R. J., Parker, L. L., Wei, A., and Tao, W. A. (2015) Time-Resolved Proteomic Visualization of Dendrimer Cellular Entry and Trafficking. J Am Chem Soc 137, 12772-12775.
53. lliuk, A. B., and Tao, W. A. (2015) Universal non-antibody detection of protein phosphorylation using pIMAGO. Curr Protoc Chem Biol 7, 17-25.
54. lliuk, A., Liu, X. S., Xue, L., Liu, X., and Tao, W. A. (2012) Chemical visualization of phosphoproteomes on membrane. Mol Cell Proteomics 11, 629-639.
55. lliuk, A., Martinez, J. S., Hall, M. C., and Tao, W. A. (2011) Phosphorylation assay based on multifunctionalized soluble nanopolymer. AnalChem 83, 2767-2774.
56. Tao, W. A., Wollscheid, B., O'Brien, R., Eng, J. K., Li, X. J., Bodenmiller, B., Watts, J. D., Hood, L., and Aebersold, R. (2005) Quantitative phosphoproteome analysis using a dendrimer conjugation chemistry and tandem mass spectrometry. Nat Methods 2, 591-598.
57. lliuk, A., and Tao, W. A. (2009) Quantitative phospho-proteomics based on soluble nanopolymers. Methods Mol Biol 527, 117-129, ix.
58. lliuk, A., Martinez, J. S., Hall, M. C., and Tao, W. A. (2011) Phosphorylation assay based on multifunctionalized soluble nanopolymer. AnalChem 83, 2767-2774.
59. Iliuk, A., Jayasundera, K., Schluttenhofer, R., and Tao, W. A. (2011) Functionalized soluble nanopolymers for phosphoproteome analysis. Methods Mol Biol 790, 277-285.
60. Xue, L., Wang, W. H., Iliuk, A., Hu, L., Galan, J. A., Yu, S., Hans, M., Geahlen, R. L., and Tao, W. A. (2012) Sensitive kinase assay linked with phosphoproteomics for identifying direct kinase substrates. Proc Natl Acad Sci USA 109, 5615-5620.
61. Xue, L., Wang, P., Wang, L., Renzi, E., Radivojac, P., Tang, H., Arnold, R., Zhu, J. K., and Tao, W. A. (2013) Quantitative measurement of phosphoproteome response to osmotic stress in arabidopsis based on Library-Assisted eXtracted Ion Chromatogram (LAXIC). Molecular & cellular proteomics: MCP 12, 2354-2369.
62. Iliuk, A. B., and Tao, W. A. (2013) Is phosphoproteomics ready for clinical research? Clinica chimica acta; international journal of clinical chemistry 420, 23-27.
63. Xue, L., Geahlen, R. L., and Tao, W. A. (2013) Identification of Direct Tyrosine Kinase Substrates Based on Protein Kinase Assay-Linked Phosphoproteomics. Molecular & cellular proteomics: MCP 12, 2969-2980.
64. Arrington, J. V., Xue, L., and Tao, W. A. (2014) Quantitation of the phosphoproteome using the library-assisted extracted ion chromatogram (LAXIC) strategy. Methods Mol Biol 1156, 407-416.
65. Xue, L., Wang, P., Cao, P., Zhu, J. K., and Tao, W. A. (2014) Identification of extracellular signal-regulated kinase 1 (ERK1) direct substrates using stable isotope labeled kinase assay-linked phosphoproteomics. Molecular & cellular proteomics: MCP 13, 3199-3210.
66. Iliuk, A., Jayasundera, K., Wang, W. H., Schluttenhofer, R., Geahlen, R. L., and Tao, W. A. (2015) In-Depth Analyses of B Cell Signaling Through Tandem Mass Spectrometry of Phosphopeptides Enriched by PolyMAC. International journal of mass spectrometry 377, 744-753.
67. Jayasundera, K. B., Iliuk, A. B., Nguyen, A., Higgins, R., Geahlen, R. L., and Tao, W. A. (2014) Global phosphoproteomics of activated B cells using complementary metal ion functionalized soluble nanopolymers. Anal Chem 86, 6363-6371.
68. Pan, L., Wang, L., Hsu, C. C., Zhang, J., lliuk, A., and Tao, W. A. (2015) Sensitive measurement of total protein phosphorylation level in complex protein samples. The Analyst 140, 3390-3396.
69. Pan, L., lliuk, A., Yu, S., Geahlen, R. L., and Tao, W. A. (2012) Multiplexed quantitation of protein expression and phosphorylation based on functionalized soluble nanopolymers. J Am Chem Soc 134, 18201-18204.
70. lliuk, A., Li, L., Melesse, M., Hall, M. C., and Tao, W. A. (2016) Multiplexed Imaging of Protein Phosphorylation on Membranes Based on Ti(IV) Functionalized Nanopolymers. Chembiochem: a European journal of chemical biology 17, 900-903.
71. Shariat, S. F., Lotan, Y., Vickers, A., Karakiewicz, P. I., Schmitz-Drager, B. J., Goebell, P. J., and Malats, N. (2010) Statistical consideration for clinical biomarker research in bladder cancer. Urol Oncol 28, 389-400.
72. Nagata, M., Muto, S., and Horie, S. (2016) Molecular Biomarkers in Bladder Cancer: Novel Potential Indicators of Prognosis and Treatment Outcomes. Disease markers 2016, 8205836.
73. Mossanen, M., and Gore, J. L. (2014) The burden of bladder cancer care: direct and indirect costs. Current opinion in urology 24, 487-491.
74. Shariat, S. F., Karam, J. A., Lotan, Y., and Karakiewizc, P. I. (2008) Critical evaluation of urinary markers for bladder cancer detection and monitoring. Reviews in urology 10, 120-135.
75. Darwiche, F., Parekh, D. J., and Gonzalgo, M. L. (2015) Biomarkers for non-muscle invasive bladder cancer: Current tests and future promise. Indian journal of urology: IJU: journal of the Urological Society of India 31, 273-282.
76. Santoni, G., Morelli, M. B., Amantini, C., and Battelli, N. (2018) Urinary Markers in Bladder Cancer: An Update. Front Oncol 8, 362.
77. Vriesema, J. L., Poucki, M. H., Kiemeney, L. A., and Witjes, J. A. (2000) Patient opinion of urinary tests versus flexible urethrocystoscopy in follow-up examination for superficial bladder cancer: a utility analysis. Urology 56, 793-797.
78. Duffy, M. J., Sturgeon, C. M., Soletormos, G., Barak, V., Molina, R., Hayes, D. F., Diamandis, E. P., and Bossuyt, P. M. (2015) Validation of new cancer biomarkers: a position statement from the European group on tumor markers. Clinical chemistry 61, 809-820.
79. Hayes, D. F. (2015) Biomarker validation and testing. Mol Oncol 9, 960-966.
80. lliuk A., Wu X., Li L., Sun J., Hadisurya M., Boris R., Tao W. A. (2020). Plasma-Derived Extracellular Vesicle Phosphoproteomics through Chemical Affinity Purification. J Proteome Res. 19(7): 2563-2574. PMID: 32396726.

What is claimed is:

1. A compound comprising:
a biomarker for urological cancers selected from the group consisting of proteins or phosphoproteins and any combination thereof, wherein each of the proteins, phosphoproteins or their combinations are capable of differentiating a human with bladder cancer from a healthy human, a human with non-cancer inflammation, or other relevant conditions, for the purposes of bladder cancer diagnosis, prognosis, detection, monitoring, patient stratification, drug response analysis, therapy selection, or the like.

2. The compound of claim 1 wherein the biomarker has a putative compound identification, match form, name or pathway.

3. The compound of claim 1, wherein the biomarker is located on, in or about an extracellular vesicle.

4. The compound of claim 3, wherein the extracellular vesicle including the biomarker is captured, enriched or isolated using a method for capture, enrichment or isolation of extracellular vesicles.

5. The compound of claim 4, wherein the method for capture, enrichment or isolation of extracellular vesicles is selected from the group consisting of Extracellular Vesicles total recovery and purification (EVtrap), ultracentrifugation (UC), filtrations, antibody-based purification, size-exclusion approach, polymer precipitation and affinity capture.

6. The compound of claim 3, wherein the biomarker is detected from urine.

7. The compound of claim 6, wherein the biomarker is selected from a pre-determined biomarkers panel.

8. The compound of claim 3, wherein the extracellular vesicle is an exosome, an endosome, a microvesicle or the like.

9. A method of detecting biomarkers comprising the steps of:
analyzing urine from humans with bladder cancer, healthy humans, humans with non-cancer inflammation, or other relevant conditions for an extracellular vesicle (EV) biomarker; and
detecting a biomarker in each urine samples for the purposes of bladder cancer diagnosis, prognosis, detection, monitoring, patient stratification, drug response analysis, therapy selection, or the like, wherein the biomarker is selected from the group consisting of proteins or phosphoproteins and any combination thereof.

10. The method of claim 9, further comprising the step of:
analyzing differences in the detected biomarkers between cancer and non-cancer urine samples including observing that an EV proteomics of humans having bladder cancer has clear separation from an EV proteomics of humans having non-cancer inflammation or healthy controls.

11. The method of claim 10, further comprising the step of:
assessing a disease predictive capacity of the detected biomarkers.

12. The method of claim 11, further comprising the step of:
identification of novel biomarkers.

13. The method of claim 9, wherein the biomarkers are selected from a pre-determined biomarkers panel.

14. A method of detecting biomarkers comprising the steps of:
isolating and capturing extracellular vesicles (EVs) from urine samples from humans with bladder cancer, healthy humans (controls), humans with non-cancer inflammation, or other relevant conditions for an EV biomarker, wherein the biomarker is selected from the group consisting of proteins or phosphoproteins and any combination thereof;
analyzing the isolated and captured EVs by liquid chromatography-mass spectrometry, wherein the analysis step provides an EV protein profile (EV proteomics) for each urine sample; and
analyzing differences of the EV proteomics of humans having bladder cancer and of the EV proteomics of humans having non-cancer inflammation or healthy controls.

15. The method of claim 14, further comprising the step of:
processing and enrichment of the isolated and captured EVs prior to the liquid chromatography-mass spectrometry, filtering out soluble proteins and retaining EV associated proteins.

16. The method of claim 14, further comprising the step of:
performing biostatistical analysis in detected biomarkers between cancer and non-cancer controls including observing that the EV proteomics of humans having bladder cancer has clear separation from the EV proteomics of humans having non-cancer inflammation or healthy controls.

17. The method of claim 16, further comprising the step of:
assessing a disease predictive capacity of detected biomarkers.

18. The method of claim 17, further comprising the step of:
identification of novel biomarkers.

19. The method of claim 14, wherein the biomarkers are selected from a pre-determined biomarkers panel.
